(19) United States
(12) Patent Application Publication
(10) Pub. No.: US 2018/0143207 A1
(43) Pub. Date: May 24, 2018

(54) METHOD FOR SCREENING DIABETIC NEPHROPATHY IN A SUBJECT

(71) Applicant: Changhua Christian Medical Foundation Changhua Christian Hospital, Changhua (TW)

(72) Inventors: CHUNG HO CHANG, Changhua (TW); CHIA CHU CHANG, Changhua (TW)

(21) Appl. No.: 15/821,578

(22) Filed: Nov. 22, 2017

(30) Foreign Application Priority Data
Nov. 23, 2016 (TW) 105138384

(51) Int. Cl. G01N 33/68; G01N 33/70

(52) U.S. Cl. CPC G01N 33/6893; G01N 33/70; G01N 2333/4703; G01N 2333/76; G01N 2440/38; G01N 2800/347; G01N 2800/042

(57) ABSTRACT

A urine biomarker is provided, and more particularly, a screening method using the urine biomarker to screen a subject having a high risk of developing diabetic nephropathy is also provided. The urine biomarker of glycosylated uromodulin can be used to detect and predict diabetic patients having a high risk of developing diabetic nephropathy more accurate and earlier than general detection methods of PCR or ACR tests, thereby the patient being screened can be treated earlier and thus prevent deterioration of the progression of chronic kidney disease, and therefore dramatically decrease the risk of death.

### CROSS-REFERENCES TO RELATED APPLICATIONS

This application claims priority to Taiwan Patent Application No. 105138384 filed on 23 Nov. 2016, all disclosures of which are incorporated herein by reference in its entirety.

### BACKGROUND OF THE INVENTION

### 1. Field of the Invention

The present invention relates generally to a urinary biomarker, and more particularly to a method for screening out a subject having a high risk of developing a diabetic nephropathy by the urinary biomarker.

### 2. The Prior Arts

Although the medical science is highly developed in the twenty-first century, the prevalence of diabetes or early-stage of diabetes in the world is continuously raising. In 2013, the global diabetic patients were about 382 million, and it was estimated to about 592 million by 2035, equivalent to 10.1% population of 20 to 79-year-old adult of the world. One of the most common risk factors for chronic kidney disease (CKD) is diabetes, and the major complication of diabetes, diabetic nephropathy (DN), would often lead to the serious end-stage renal disease (ESRD). The patients suffering from the ESRD require hemodialysis or renal transplantation therapy, and are always facing the death threats. According to the United States The Third National Health and Nutrition Examination survey (NHANES III), the 10-year cumulative all-cause mortality of the diabetic adults, more than 20 yeas old, increased by 3.4 times, while CKD patients increased by 9 times; however, when a diabetic patient having CKD, the all-cause mortality increased significantly to 23.4 times. Apparently, diabetic nephropathy has become the most serious threat to diabetic patients.

Even aware of the trend described above, the need of renal transplantation for the patients with end-stage of the kidney disease has still been increasing. Up to now there has no effective strategy to prevent diabetic renal complications; and the knowledge of pathogenic mechanisms of DN remains insufficient so that the method for effectively screening or predicting patients having a high risk of developing ESRD is also deficient. There are some conventional clinical screening methods for diabetic nephropathy, such as detection of albumin-creatinine ratio (ACR) or protein-creatinine ratio (PCR) in urine of patients with abnormal blood glucose. As the value of ACR or PCR is much higher, the patient's renal function may be decreased more significantly in the future. Furthermore, detection of microalbuminuria has been the standard method for diagnosis of early stages of DN. However, some patients detected with microalbuminuria have appeared advanced renal pathological changes, which indicates that microalbuminuria is not an appropriate marker for early detection or prediction of DN. In addition, some patients with type 2 diabetes have found no microalbumin appeared after renal biopsy; that is, the pathology of part of diabetic patients is atypical diabetic nephropathy, and particularly some of them exhibit both non-diabetic nephropathy and diabetic nephropathy. As such, detection of microalbuminuria is therefore limited. Moreover, the prevalence or incidence of microalbuminuria in Asia patients with diabetes is higher than that of Caucasians, and so is the rate of deterioration of nephropathy. Therefore, it is urgently needed to find a suitable biomarker for precise detection of early diabetic nephropathy such that the diabetic patients having high risk of developing DN can be screened and treated earlier.

As mentioned above, the pathogenesis of diabetic nephropathy is unclear, but a variety of studies have shown that blood glucose abnormalities play an important role. In the case of patients with type 1 diabetes, strict glycemic control can reduce the probability of albuminuria and deterioration thereof. In addition to abnormal blood glucose, the most relevant factor regarding diabetic nephropathy is advanced glycation end products (AGEs). The advanced glycation end products are formed through a series of Maillard reactions, reducing sugars, such as glucose, react with amino groups in proteins, lipids, and nucleic acids forming Schiff bases and Amadori products to produce AGEs. In general, AGEs are produced continuously in the human body, even with euglycemia, but in diabetes patients AGEs are accelerated produced. The resultant AGEs are finally removed or metabolized by the kidney in normal subjects. However, AGEs are markedly accumulated in the serum and tissues of patients with ESRD. Compared with diabetic patients without renal disease, diabetic patients with ESRD had two times of AGE levels in tissues.

Uromodulin (UMOD), also known as Tamm-Horsfall protein, is the most abundant urinary protein in healthy individuals and normally expressed by epithelia of the TALH and the early distal tubule. UMOD exhibits diverse functions including the prevention of ascending urinary tract infections by binding type I-fimbriated Escherichia coli, up-regulation inflammatory response and tubular transport function. UMOD, normal and genetically determined variants, may also actively participate in the pathogenesis of CKD. As loss of protective UMOD activities, gain of damaging functions or dislocation, UMOD might impair tubular recovery after injury and promote chronic interstitial fibrosis and irreversible nephron loss due to abnormal intracellular trafficking, leading to stress within the endoplasmic reticulum (ER), tubular malfunction and eventual death. Many studies have confirmed the findings that there was association between UMOD with eGFR and diabetic nephropathy.

### SUMMARY OF THE INVENTION

A primary objective of the present invention is to provide a urinary biomarker for early detection or prediction of a patient having a high risk of developing a diabetic nephropathy so as to diagnose and treat diabetic nephropathy earlier to decrease the probability of serious complications and the mortality resulting from ESRD.

In order to achieve the foregoing objective, the present invention provides a urinary biomarker, which can be used to screen the patients having high risks of developing a diabetic nephropathy. The urinary biomarker can include glycated uromodulin (glcUMOD).

Furthermore, in order to achieve the foregoing objective, the present invention also provides a method for evaluating a subject having a high risk of developing a diabetic nephropathy, comprising the steps of: providing a urine sample from the subject; and detecting the presence of glycated uromodulin in the urine sample. When glycated uromodulin is present in the urine sample, the subject is diagnosed to have a high risk of developing a diabetic nephropathy.

According to an embodiment of the present invention, the subject for evaluating is a diabetic patient. According to an embodiment of the present invention, the age of the subject can be less than 65 years old.

According to an embodiment of the present invention, the subject can be patients of stages 1 to 3a of CKD.

According to an embodiment of the present invention, the urine sample can be obtained from the supernatant fraction of the urine sample centrifugation.

According to an embodiment of the present invention, the urine sample can be centrifuged at about 16,000 xg to about 20,000 xg, preferably at about 18,000 xg, and then the supernatant is recovered. The supernatant recovered can further be centrifuged at about 100,000 xg to about 120,000 xg, preferably at 110,000 xg, and the resultant supernatant is further recovered.

According to an embodiment of the present invention, the level of glycated uromodulin can be determined by, including but not limited to, Western blot, mass spectrometry, immunoassay, or chromatography.

According to an embodiment of the present invention, the level of glycated uromodulin in the urine sample can be 8,000 a.u. (arbitrary unit) or more, preferably 9,000 a.u. or more.

Accordingly, the biomarker and the detection method provided by the present invention, compared to detection of microalbuminuria, PCR or ACR, can earlier and precisely screen out the diabetic patients having high risks of developing a diabetic nephropathy so that these patients can be treated in advance to prevent the progression of ESRD and decrease the subsequent pain of hemodialysis or probable death.

Furthermore, according to an embodiment of the present invention, when the subject is detected to have the glycated uromodulin in the urine sample, especially the level of glycated uromodulin is more than 8,000 a.u., preferably 9,000 a.u., An albumin-creatinine ratio (ACR) or a protein-creatinine ratio (PCR) of the urine sample can be furthered detected so as to increase the accuracy the prediction of DN.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a Western blot analysis of glycated uromodulin isolated and determined from the urine sample of patients according to an embodiment of the present invention;

FIG. 2 is a Western blot analysis showing glycated uromodulin is mostly excreted in the supernatant fraction of the urine sample from the diabetic patients according to an embodiment of the present invention;

FIG. 3 shows the median levels of urine glycated uromodulin expression in diabetic CKD patients and non-diabetic CKD patients according to an embodiment of the present invention;

FIG. 4 shows the levels of urine glycated uromodulin expression in diabetic patients of early CKD stage and advanced CKD stage according to an embodiment of the present invention;

FIG. 5 shows the correlation between urine glycated uromodulin levels and the probability of diabetic CKD; and

FIG. 6 is an AUC-ROC analysis of protein-creatinine ratio (PCR), albumin-creatinine ratio (ACR), and glycated uromodulin (glcUMOD) showing the correlation of diabetic nephropathy therebetween.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

In order to isolate and determine the glycated uromodulin, the AGEs isolated from urine of non-diabetic CKD patients (non-DM) and diabetic CKD patients (DM) were immunoprecipitated and then the immunoprecipitates were subjected to LC-MS-MS, and further confirmed by Western blot analysis. Finally, glycated uromodulin was mainly present in the urine of DM subjects, especially in the supernatant fraction of urine thereof.

### Example 1 Isolation and Determination of Glycated Uromodulin

Firstly, 84 patients, 35 DM and 49 non-DM, were recruited from the nephrology clinic at Changhua Christian Hospital, Taiwan, between September 2013 and January 2015. The duration of follow-up was less than 3 years in all patients. Those with fever, infection, hepatic, cardiac, history of other endocrinopathies, surgery, trauma and admission for any causes in recent 3 months were excluded. The study protocol was approved by the Ethics Research Committee at Changhua Christian Hospital, Taiwan). Written informed consent was obtained from all participants. Next, these patients were fast overnight for 8 hours. Then, venous blood samples thereof were obtained and the first morning urine samples were also collected from each individual. After that, aliquots of urine were immediately frozen at −80° C. until further analysis.

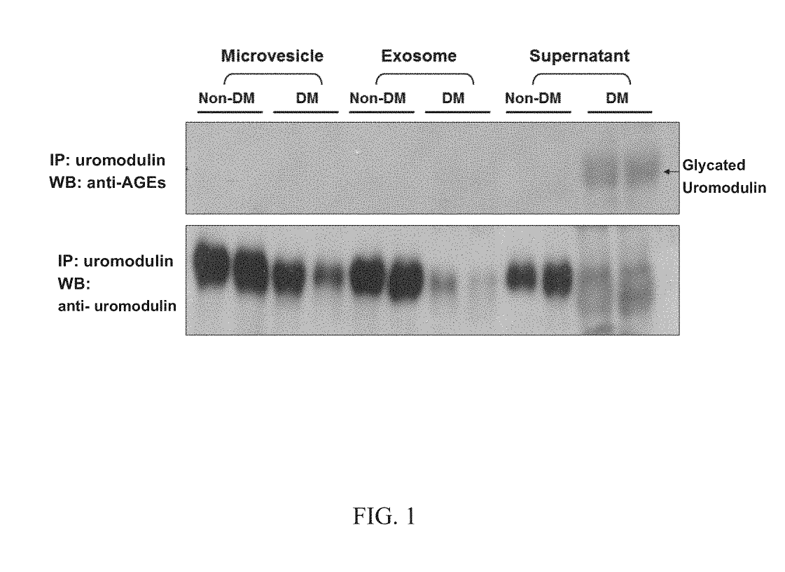

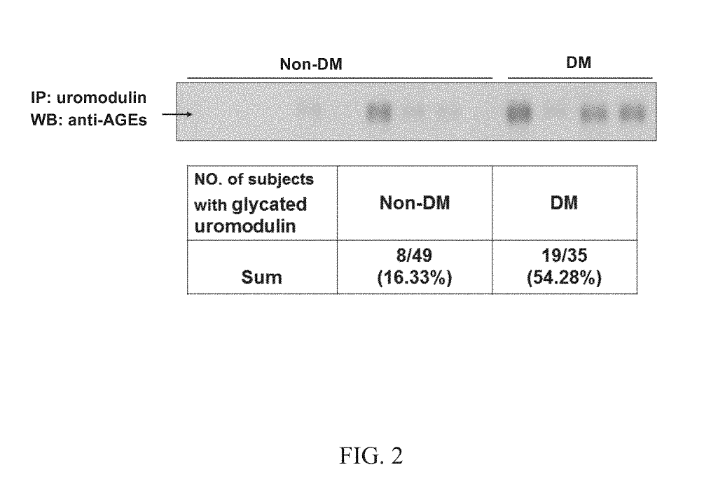

In order to isolate glycated uromodulin, the urine samples were thawed and then isolated by differential centrifugation at 4° C., 18,000 xg for 3 hours. The supernatant collected therefrom was further centrifuged at 4° C., 111,000 xg for 3 hours. After that, the resultant supernatant and pellet (including exosome), and the pellet (including microvescile) obtained by the first centrifugation were immunoprecipitated with anti-uromodulin antibodies (IP: uromodulin) and subjected to LC-MS-MS. Next, the immunoprecipitates were further confirmed respectively by Western blot analysis with anti-AGEs and anti-uromodulin antibodies (WB: anti-AGEs and WB: anti-uromodulin). As the results shown in FIG. 1, glycated uromodulin is present in the supernatant of urine (i.e., secreted via the classical protein secretion pathway). By similar steps, the urine samples were first centrifuged at 4° C., 18,000 xg for 3 hours, and the supernatant obtained was then immunoprecipitated by anti-uromodulin antibodies and confirmed by Western blot analysis with anti-AGEs antibodies. As the results shown in FIG. 2, glycated uromodulin is mostly excreted in the supernatant fraction of diabetic patients' urine. The method for determination or quantitation of glycated uromodulin may not limit to the method described above, which may include any methods known to a person skilled in the art, such as mass spectrometry, immunoassay (e.g. ELISA), or chromatography.

Referring to FIG. 1 and FIG. 2, the glycated uromodulin is mainly present in the urine of diabetic CKD (DM) patients, and the percentage of DM patients having the glycated uromodulin to all DM patients is 54.28%. However, there are only 16.33% of non-diabetic CKD (Non-DM) patients found to have the glycated uromodulin. Especially, the glycated uromodulin is present in the supernatant fraction of urine been centrifuged; thereby, the present invention provides a special biomarker in the urine for discriminate diabetic CKD patients from non-diabetic CKD patients so that the glycated uromodulin, compared to generally present uromodulin, can serve as an indicator to predict a diabetic patient has a high probability to progress to DN.

In addition to glycated uromodulin determination, microalbuminuria was established when two out of three ACR determinations were found to be within the range 30-300 mg/g in a six-month period. Creatinine concentration in urine and serum was measured by a kinetic method based on Jaffe reaction. The tests were run in duplicates. Intra-assay variation coefficient was <5%. The urinary concentration of albumin was assessed by an immunoturbidimetric method (Roche Diagnostics GmbH) and ACR was expressed as mg/g creatinine. Serum creatinine values were used to calculate an estimated glomerular filtration rate (eGFR), by means of the abbreviated Modification of Diet in Renal Disease formula.

Results from above tests were presented as the median (interquartile range) or percentage N (%). Statistical analyses were performed using Chi-Square test or Fisher's Exact test for comparing the proportion of categories variables of serum glcUMOD levels between patients with and without DM. As for nonparametric Wilcoxon rank-sum test was employed to compare the continuous variable of serum glcUMOD levels between patients with or without DM. The predicted probability of DM at various glcUMOD levels was calculated from a logistic regression model. Statistical analyses were performed with SPSS Statistics software version 19.0.0 (IBM Corporation, Somers, N.Y., USA). P<0.05 was considered to be statistically significant.

Referring to TABLE 1 below, it summarizes some clinical features of the study subjects, and also the glcUMOD levels and eGFR of patients. CKD patients can be classified into five stages by values of eGFR as follows: (1) Stage 1: Slightly diminished function; kidney damage with normal or relatively high eGFR (>90 ml/min/1.73 m²) and persistent albuminuria. Kidney damage is defined as pathological abnormalities or markers of damage, including abnormalities in blood or urine tests or imaging studies; (2) Stage 2: Mild reduction in eGFR (60-89 ml/min/1.73 m²) with kidney damage; (3) Stage 3: Moderate reduction in eGFR (30-59 ml/min/1.73 m²; it can further be divided into stage 3a having eGFR of 45-59 ml/min/1.73 m² and stage 3b having eGFR of 30-44 ml/min/1.73 m²); (4) Stage 4: Severe reduction in eGFR (15-29 ml/min/1.73 m²). Preparation for renal replacement therapy; and (5) Stage 5: Established kidney failure (eGFR <15 ml/min/1.73 m²), permanent renal replacement therapy, or end-stage renal disease.

TABLE 1

| Total of | DM patients | | |
|---|---|---|---|
| patients | No | Yes | p-value |

Discrete variable: N(%)

| | | | | | | | |
|---|---|---|---|---|---|---|---|
| Sex | Female | 29 | 17 | (34.69%) | 12 | (34.29%) | 0.969 |
| | Male | 55 | 32 | (65.31%) | 23 | (65.71%) | |
| Stage of CKD | I | 4 | 2 | (4.08%) | 2 | (5.71%) | 0.262 |
| | II | 13 | 9 | (18.37%) | 4 | (11.43%) | |
| | IIIa | 11 | 8 | (16.33%) | 3 | (8.57%) | |
| | IIIb | 18 | 13 | (26.53%) | 5 | (14.29%) | |
| | IV | 20 | 10 | (20.41%) | 10 | (28.57%) | |
| | V | 18 | 7 | (14.29%) | 11 | (31.43%) | |

Continuous variable: Median (Q1-Q3)

| | | | | | | |
|---|---|---|---|---|---|---|
| Age (years) | 84 | 57 | (45, 61) | 60 | (55, 65) | 0.048 |
| BMI (Kg/m²) | 84 | 23 | (20, 26) | 27 | (25, 30) | 0.015 |
| glcUMOD | 84 | 0 | (0, 0) | 6027 | (0, 16820.82) | 0.000 |
| eGFR | 84 | 38.55 | (26.71, 55.58) | 23.32 | (10.64, 48.21) | 0.055 |

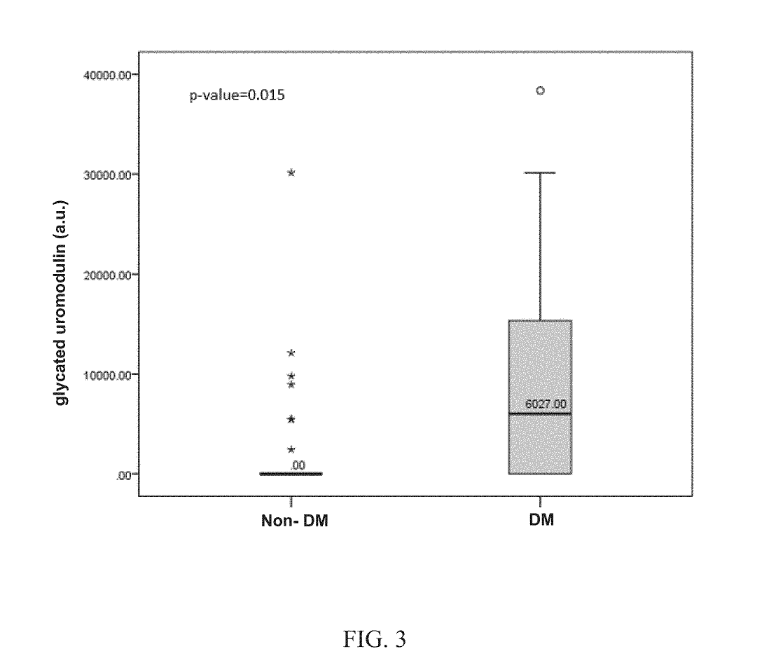

As shown in TABLE 1, the relationship via Person correlation analysis between glcUMOD and age (Sig.=0.773; 2-tailed) or body mass index (BMI) and glcUMOD (Sig.=0.059; 2-tailed) is statistic insignificancy. Referring also to FIG. 3, it shows the median levels of urine glycated uromodulin expression in patients with or without DM. As seen from FIG. 3 and TABLE 1, the median level of urine glycated uromodulin expression in patients without DM is zero. In contrast, the median level of urine glycated uromodulin expression in patients with DM is as high as 6027 a.u.; namely the glycated uromodulin has a significant correlation with patients with DM.

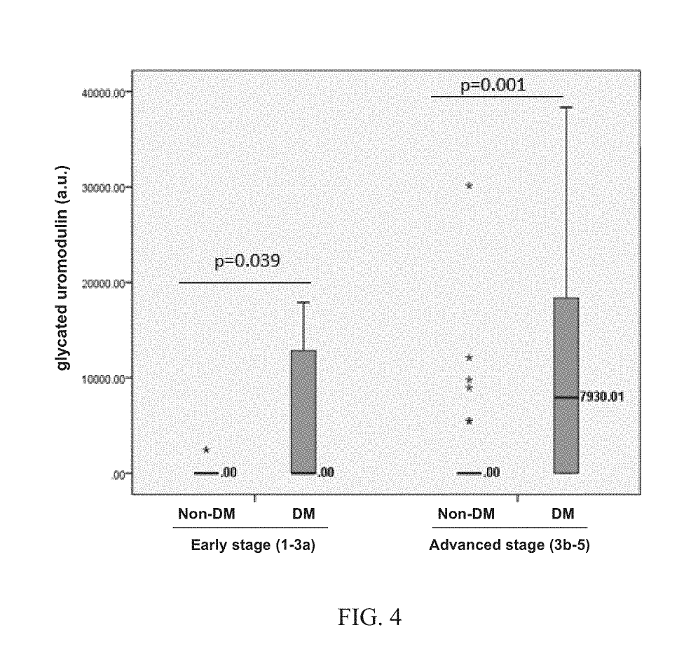

Referring to both FIG. 4 and TABLE 2 below, they show the levels of urine glycated uromodulin expression in non-diabetic and diabetic patients of early CKD stage and advanced CKD stage. As shown in FIG. 4 (right bar of early stage and advanced stage) and TABLE 2, the urine glcUMOD expression is significantly higher in diabetic patients of both early CKD stage and advanced CKD stage, and thus has a significant correlation therewith. Especially, when the level of glcUMOD is more than 9000 a.u., the subjects are mostly belonging to DM patients (about 60% positive predictive value).

TABLE 2

| | | Total | | | | |
|---|---|---|---|---|---|---|
| Stage | glcUMOD | patients | Non-DM | | DM | |
| All stages | >=9000 | 19 | 3 | (6.12%) | 16 | (45.71%) |
| of CKD | <9000 | 65 | 46 | (93.88%) | 19 | (54.29%) |
| Early stage | >=9000 | 3 | 0 | (0%) | 3 | (33.33%) |
| (Stage 1-3a) | <9000 | 25 | 19 | (100%) | 6 | (66.67%) |
| Advanced stage | >=9000 | 16 | 3 | (10%) | 13 | (50%) |
| (Stage3b-5) | <9000 | 40 | 27 | (90%) | 13 | (50%) |

Further referring to TABLE 3 below, it shows the levels of urine glycated uromodulin expression in diabetic patients having age less or more than 65 years old. As shown in TABLE 3, patients' age has a significant correlation with the diabetic patient; particularly, the correlation is more obvious when the age is younger than 65 years old.

TABLE 3

| | | Total | | | | |
|---|---|---|---|---|---|---|
| Age | glcUMOD | patients | Non-DM | | DM | |
| <65 | >=9000 | 15 | 2 | (5%) | 13 | (50%) |
| | <9000 | 51 | 38 | (95%) | 13 | (50%) |
| >=65 | >=9000 | 4 | 1 | (11.11%) | 3 | (33.33%) |
| | <9000 | 14 | 8 | (88.89%) | 6 | (66.67%) |

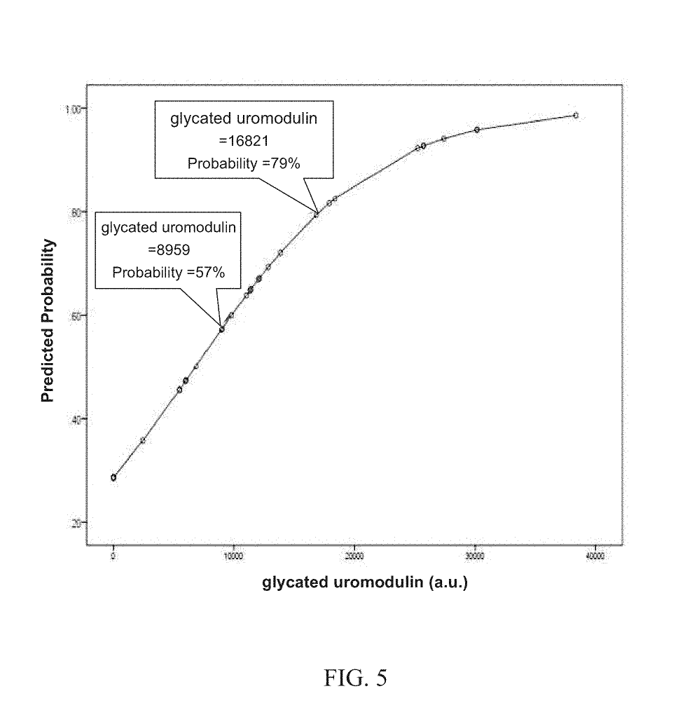

FIG. 5 shows the correlation between urine glycated uromodulin levels and the probability of diabetic CKD. As shown in FIG. 5, the higher the urine glcUMOD level, the higher the probability of suffering from diabetic CKD (e.g. when the glcUMOD is about 9000 a.u. (8,959 a.u.), the correlation between urine glycated uromodulin levels and the probability of diabetic CKD (Prob_DM) is 0.57; and when the glcUMOD is 16,821 a.u., the probability of diabetic CKD has increased to 0.79).

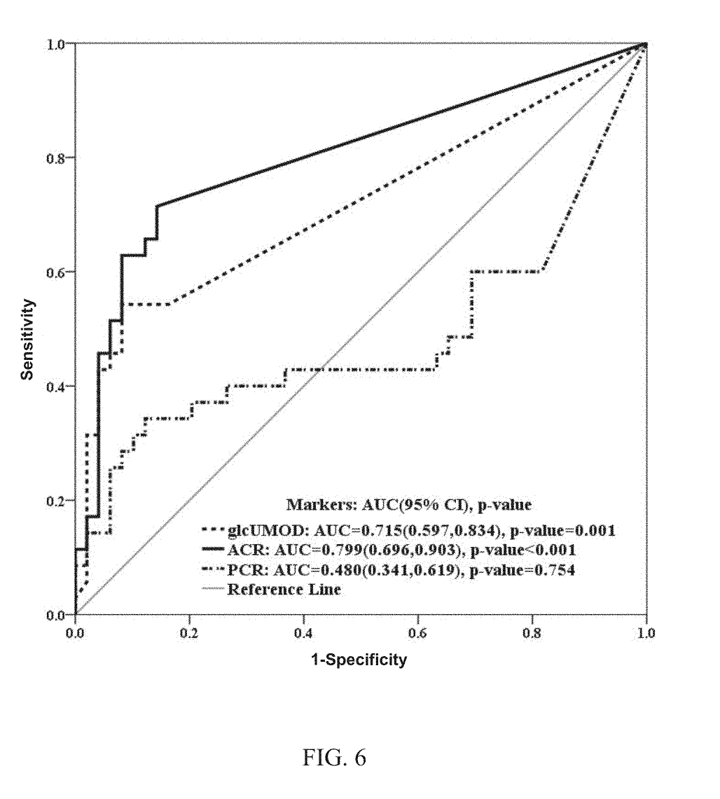

In order to determine the prediction accuracy of the biomarker of glycated uromodulin, the other well-known biomarkers, such as protein-creatinine ratio (PCR) and albumin-creatinine ratio (ACR) were performed at the same time to analyze patients who was and was not with diabetic nephropathy. Referring to FIG. 6, the AUC-ROC (Area under the Curve of ROC) of the urine glcUMOD is 0.715 (95% CI: 0.597-0.834; p-value=0.001). The AUC-ROC analysis of ACR is 0.799 (95% CI: 0.696-0.903; p-value=0.001) and AUC-ROC of PCR is 0.480 (95% CI: 0.341-0.619; p-value=0.754). Accordingly, glycated uromodulin is an excellent biomarker for detection, and its prediction accuracy is close to that of using ACR.

### Example 2 Risk Prediction Model of DN

A risk prediction model was assessed using multivariable logistic regression and predictive ability was using c-statistics, category-free net reclassification improvement (cfNRI), integrated discrimination improvement (IDI) for the urine biomarkers model. The models were shown in TABLE 4 below.

TABLE 4

| | Model 1a | | Model 1b | | Model 2a | | Model 2b | |
|---|---|---|---|---|---|---|---|---|
| Markers* | OR | p-value | OR | p-value | OR | p-value | OR | p-value |
| ACR | 1.48 (1.25, 1.74) | <0.0001 | 1.45 (1.20, 1.75) | <0.0001 | | | | |
| glcUMOD | | | 1.14 (1.01, 1.29) | 0.028 | | | 1.23 (1.11, 1.38) | <0.0001 |
| PCR | | | | | 0.94 (0.83, 1.06) | 0.325 | 0.88 (0.77, 1.02) | 0.092 |
| vif | | | 1.105 | | | | 1.022 | |

*The markers were transformed by log(marker + 0.5) from [0, 1000000] to real line.

Following ACR (Model 1a) or ACR and glcUMOD (Model 1b) adjustment, the glcUMOD is predictive for diabetic patients with CKD (odds ratio 1.14 (95% CI: 1.01-1.29), P=0.028); as PCR (Model 2a) or PCR and glcUMOD (Model 2b) adjustment, the glcUMOD is well predictive for diabetic patients with CKD (odds ratio 1.23 (95% CI: 1.11-1.38), P<0.0001). Variance inflation factors (vif) of the logistic regression were 1.105 and 1.022 respectively, indicating there are absent the collinearity between ACR and glcUMOD, PCR and glcUMOD.

To further describe the ability of these markers to risk-stratify diabetic patients beyond our clinical classic risk prediction model, c-statistics, NRI, IDI values were calculated and the results are shown in TABLE 5.

TABLE 5

| | | Change of | | | | | |
|---|---|---|---|---|---|---|---|
| | C statistic | C statistic | | cfNRI(%) | | IDI | |
| Markers | (95% CI) | (95% CI) | p-value | (95% CI) | p-value | (95% CI) | p-value |
| Proteinuria | 0.799 (0.70, 0.90) | — | Referent | — | Referent | — | Referent |
| ACR + glcUMOD | 0.867 (0.78, 0.95) | 0.068 (−0.06, 0.20) | 0.311 | 75.92 (36.96, 114.88) | <0.0001 | 0.046 (0.002, 0.09) | 0.048 |
| Proteinuria | 0.520 (0.39, 0.65) | — | Referent | — | Referent | — | Referent |
| PCR + glcUMOD | 0.746 (0.64, 0.86) | 0.226 (0.06, 0.39) | 0.008 | 75.92 (36.96, 114.88) | <0.0001 | 0.190 (0.103, 0.277) | <0.0001 |

The IDI provides information on both the direction and magnitude of mean change in predicted probabilities for events and nonevents when additional variables or biomarkers are added. Referring to TABLE 5, as urine glcUMOD combing ACR, the IDI was 0.046 (95% CI: 0.002-0.09), P=0.048; namely the prediction probability increases for 4.6%. Whereas glcUMOD combing PCR, the IDI was 0.19 (95% CI: 0.103-0.277), P<0.0001). That is, the prediction probability increases for 19%.

In contrast, cfNRI provides a measure of the direction of change in estimated risk that a biomarker adds to the clinical model without considering cut-point exist, with results reported as proportions. Because it is possible for 100% of both events and nonevents to increase the risk, the maximum value of the total cfNRI (events+ nonevents) is 200%. As seen from TABLE 5, as urine glcUMOD combing ACR, the cfNRI increases 75.92% (95% CI: 36.96-114.88), P<0.0001). Whereas glcUMOD combing PCR, the cfNRI increases also 75.92% (95% CI: 36.96-114.88), P<0.0001). Therefore, combing the detection results of glcUMOD according to an embodiment of the present invention with the results of conventional ACR or PCR would further increase the prediction accuracy of DN.

By means of using glycosylated uromodulin as a biomarker, patients with high risks of developing diabetic nephropathy can be predicted accurately and earlier more than general detection methods of PCR or ACR tests in diabetic patient population. Thereby the patient being screened can be treated earlier and thus prevent deterioration of the progression of chronic kidney disease, and therefore dramatically decrease the risk of death.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A method for evaluating a subject having a high risk of developing a diabetic nephropathy, comprising the steps of:
providing a urine sample from the subject; and
detecting the presence of glycated uromodulin in the urine sample.

2. The method according to claim 1, wherein the subject is a diabetic patient.

3. The method according to claim 2, wherein the age of the subject is less than 65 years old.

4. The method according to claim 1, wherein the urine sample is obtained from the supernatant fraction of the urine sample centrifugation.

5. The method according to claim 4, wherein the urine sample is centrifuged at about 16,000×g to about 120,000×g.

6. The method according to claim 1, wherein the level of glycated uromodulin is determined by Western blotting, mass spectrometry, immunoassay, or chromatography.

7. The method according to claim 1, wherein the level of glycated uromodulin in the urine sample is 9,000 a.u. or more.

8. The method according to claim 7, wherein an albumin-creatinine ratio (ACR) or a protein-creatinine ratio (PCR) of the urine sample is further detected.
